(19) United States
(12) Patent Application Publication
(10) Pub. No.: US 2023/0233742 A1
(43) Pub. Date: Jul. 27, 2023

(54) DRUG-LOADED MEDICAL DEVICE AND PREPARATION METHOD, DRUG BALLOON, AND DRUG COATING PREPARATION METHOD

(71) Applicant: SHANGHAI MICROPORT MEDICAL (GROUP) CO., LTD., Shanghai (CN)

(72) Inventors: Meng LI, Shanghai (CN); Yufu WANG, Shanghai (CN); Junfei LI, Shanghai (CN); Yan HU, Shanghai (CN)

(21) Appl. No.: 18/010,653

(22) PCT Filed: Jun. 18, 2021
(86) PCT No.: PCT/CN2021/101090

§ 371 Date: Dec. 15, 2022

(30) Foreign Application Priority Data
Jun. 15, 2020 (CN) 202010544839.7

(51) Int. Cl. A61L 29/16; A61L 29/08; A61L 29/14; A61L 29/18

(52) U.S. Cl. CPC A61L 29/16; A61L 29/085; A61L 29/146; A61L 29/143; A61L 29/18; A61L 2420/02; A61L 2420/06; A61L 2400/12; A61L 2300/608

(57) ABSTRACT

A drug-loaded medical device, a preparation method therefor, a drug balloon and a method of preparing a drug coating are disclosed. The medical device or the drug balloon is provided on a surface thereof with a drug coating including a stabilizer and a drug. The stabilizer includes an amphiphilic triblock polymer with hydrophilic segments at both terminals, and the drug coating forms a nano-drug particle suspension in a water-soluble environment. In this way, the prepared nano-drug coating has high drug loading and can deliver the drug in a desirable way. In particular, when it comes into contact with water, the drug can be restored to the original nano size, almost without any particle size increase. This not only avoids the risk of embolism caused by granules, but also enables higher device safety, increased drug uptake and improved therapeutic effects.

### TECHNICAL FIELD

The present invention relates to the field of medical devices and, in particular, to a drug-loaded medical device, a method of preparing the drug-loaded medical device, a drug balloon and a method of preparing a drug coating.

### BACKGROUND

Cardiovascular diseases are the number one cause of death in the world, and coronary atherosclerotic heart disease (CAHD) is one of the most mortal cardiovascular diseases and therefore poses a grave threat to human lives and health.

According to a World Health Organization (WHO) report, the number of deaths from cardiovascular diseases in the developed countries will increase by 1 million in the period from 2000 to 2020, from 5 million to 6 million. In the same period, the number of deaths from cardiovascular diseases in the low- and middle-income countries will increase by 9 million, from 10 million to 19 million. Therefore, the prevention and treatment of cardiovascular diseases has increasingly become a focus of attention of physicians from all over the world. Since the 1970s, interventional medical devices have become more and more common in the treatment of various cardiovascular diseases and have rapidly developed the three milestones: percutaneous transluminal coronary angioplasty (PTCA), bare metal stents (BMS) and drug-eluting stents (DES). In particular, the great success of drug-coated stents in the treatment of vascular stenosis has demonstrated DES' potential in use in this application. Since SeQuent® Please from B. Braun (Germany) was made available on the market in 2004, drug balloons (DCB), as a new interventional technique, have been proven by many clinical trials to be a therapeutically effective and safe treatment approach for coronary artery stenosis, small vessel disease, bifurcations and many other coronary arterial abnormalities. When a drug balloon is delivered to a target lesion site, an anti-proliferative drug uniformly coated on its surface can be released during expansion of the drug balloon which takes a short period of time (30-60 s) to inhibit the proliferation of vascular smooth muscle cells. Drug balloons are attracting more and more attention thanks to their advantages including interventional devices that do not need to be implanted, no risk of thrombosis and rapid therapeutic effects. However, existing drug balloons are associated with a number of drawbacks including significant loss during delivery, proneness to peeling off during expansion in the form of large granules which may cause embolism, and insufficient safety.

Recently, with the rapid development of nanotechnology, remarkable achievements have been made and a wealth of know-how has been accumulated in the treatment of tumors with nano-drugs. Nano-drug carriers, or nanoparticles, are usually sized in the submicron range (1-1000 nm), and materials for preparing them are principally polymers (polymeric nanoparticles, micelles or dendrimers), liposomes, viral nanoparticles and organometallic compounds. Commonly used nano-drug carriers include micelles, polymeric nanoparticles, dendrimers and liposomes. Nano-drug carriers can utilize passive and active targeting strategies to enhance the enrichment of anticancer drugs at targeted tumor sites. Nano-drug particles have a wide range of advantages including enhanced cell penetration, high drug loading capacities, sustained release, local retention and prevention of drug degradation. Using drug nanoparticles in drug balloons can result in greatly enhanced safety because their nanometer size can avoid the problem of peeling off in the form of larger particles that may cause embolism arising from the use of conventional drug coatings. It can be said that nano-drug particles are ideal for drug coating of drug balloons. However, nano-drugs reported so far can rarely be restored to their nano forms when applied to balloons and still suffer from peeling off in the form of lumps of piled-up particles that tend to cause embolism.

### SUMMARY OF THE INVENTION

It is an objective of the present invention to provide a drug-loaded medical device, a method of preparing the drug-loaded medical device, a drug balloon and a method of preparing a drug coating, in order to overcome the problems including significant loss during delivery and proneness to falling off of particles from the drug coating during expansion, which may cause embolism.

The above objective is attached by a drug-loaded medical device provided in the present invention, which has a drug coating on a surface thereof The drug coating includes a stabilizer and a drug. The stabilizer includes an amphiphilic triblock polymer with hydrophilic segments at both terminals. The drug coating forms a nano-drug particle suspension in a water-soluble environment.

Optionally, in the drug-loaded medical device, the drug coating may further include a hydrophilic spacer including a contrast agent and/or a lyoprotectant.

Optionally, in the drug-loaded medical device, the contrast agent may be selected from one or more of iohexol, iopamidol, iopromide, ioversol, iodixanol and iotrolan, and
the lyoprotectant from one or more of a saccharide, a polyhydroxy compound, an amino acid, a polymer and an inorganic salt.

Optionally, in the drug-loaded medical device, the saccharide may be selected from one or more of sucrose, trehalose, mannitol, lactose, glucose and maltose,
the polyhydroxy compound from one or more of glycerol, sorbitol, inositol and thiol,
the amino acid from one or more of proline, tryptophan, sodium glutamate, alanine, glycine, lysine hydrochloride, sarcosine, L-tyrosine, phenylalanine and arginine,
the polymer from one or more of polyvinylpyrrolidone, gelatin, polyethyleneimine, glucan, polyethylene glycol, Tween 80 and bovine serum albumin, and
the inorganic salt from one or more of a phosphate, an acetate and a citrate.

Optionally, in the drug-loaded medical device, the amphiphilic triblock polymer with hydrophilic segments at both terminals may be an ABA-type amphiphilic triblock polymer and/or an ABC-type amphiphilic triblock polymer,
where the polymeric block components A and C both include a hydrophilic group and the polymeric block component B includes a hydrophilic group.

Optionally, in the drug-loaded medical device, the polymeric block components A and C may be both from any one of the following materials: polyethylene glycol, polyvinyl alcohol, polyvinylpyrrolidone, polyether, polyester, polyamide, polypeptide and polysaccharide.

Additionally or alternatively, the polymeric block component B may be from any one of the following materials: polyoxypropylene, polycaprolactone, polylactic acid and poly(lactic-co-glycolic acid).

Optionally, in the drug-loaded medical device, the polymeric block component A or C may be from a charged hydrophilic polymer.

Optionally, in the drug-loaded medical device, the ABA-type amphiphilic triblock polymer may be selected from one or more of the following materials: poloxamer and polyethylene glycol-polycaprolactone-polyethylene glycol.

Additionally or alternatively, the ABC-type amphiphilic triblock polymer may be selected from one or more of the following materials: polyethylene glycol-polycaprolactone-glucan and polyethylene glycol-polycaprolactone-polyvinylpyrrolidone.

Optionally, in the drug-loaded medical device, the drug may include a crystalline drug and/or an amorphous drug.

Optionally, the drug-loaded medical device may further include a porous film covering the drug coating.

The above object is also attached by a drug balloon provided in the present invention, which includes a balloon body and provided on a surface of the balloon body, a drug coating and a porous film layer. The drug coating includes a stabilizer and a drug. The stabilizer includes an amphiphilic triblock polymer with hydrophilic segments at both terminals. The drug coating forms a nano-drug particle suspension in a water-soluble environment.

Optionally, in the drug balloon, the drug coating may further include a hydrophilic spacer including a contrast agent and/or a lyoprotectant.

Optionally, in the drug balloon, the stabilizer may be poloxamer. Additionally or alternatively, the contrast agent may be iopamidol. Additionally or alternatively, the drug may include paclitaxel, sirolimus or a derivative thereof Additionally or alternatively, the lyoprotectant may include one or more of a saccharide, a polyhydroxy compound, an amino acid, a polymer and an inorganic salt.

Optionally, in the drug balloon, the saccharide may be selected from one or more of sucrose, trehalose, mannitol, lactose, glucose and maltose,
the polyhydroxy compound from one or more of glycerol, sorbitol, inositol and thiol,
the amino acid from one or more of proline, tryptophan, sodium glutamate, alanine, glycine, lysine hydrochloride, sarcosine, L-tyrosine, phenylalanine and arginine,
the polymer from one or more of polyvinylpyrrolidone, gelatin, polyethyleneimine, glucan, polyethylene glycol, Tween 80 and bovine serum albumin, and
the inorganic salt from one or more of a phosphate, an acetate and a citrate.

Optionally, in the drug balloon, poloxamer and iopamidol may be present at a weight ratio of 1:0.1 to 1:10.

The above object is also attached by a method of preparing a drug-loaded medical device provided in the present invention, which includes:
obtaining a raw material of a drug coating, the raw material including a stabilizer and a drug, the stabilizer and the drug forming a nano-drug particle suspension in a water-soluble environment;
preparing the drug-loaded medical device by forming the drug coating of the raw material on a surface of a medical device; and
providing a porous film layer on a surface of the drug coating.

The above object is also attached by a method of preparing a drug coating provided in the present invention, which includes:
obtaining a raw material of the drug coating, the raw material including a stabilizer and a drug, the stabilizer and the drug forming a nano-drug particle suspension in a water-soluble environment; and
forming the drug coating of the raw material on a surface of a medical device,
wherein the stabilizer includes an amphiphilic triblock polymer with hydrophilic segments at both terminals.

Optionally, in the method, the raw material may further include a hydrophilic spacer including a contrast agent and/or a lyoprotectant.

Optionally, in the method, the contrast agent may be selected from one or more of iohexol, iopamidol, iopromide, ioversol, iodixanol and iotrolan, and the lyoprotectant from one or more of a saccharide, a polyhydroxy compound, an amino acid, a polymer and an inorganic salt.

Optionally, in the method, the saccharide may be selected from one or more of sucrose, trehalose, mannitol, lactose, glucose and maltose,
the polyhydroxy compound from one or more of glycerol, sorbitol, inositol and thiol,
the amino acid from one or more of proline, tryptophan, sodium glutamate, alanine, glycine, lysine hydrochloride, sarcosine, L-tyrosine, phenylalanine and arginine,
the polymer from one or more of polyvinylpyrrolidone, gelatin, polyethyleneimine, glucan, polyethylene glycol, Tween 80 and bovine serum albumin, and
the inorganic salt from one or more of a phosphate, an acetate and a citrate.

Optionally, in the method, the amphiphilic triblock polymer with hydrophilic segments at both terminals may be an ABA-type amphiphilic triblock polymer and/or an ABC-type amphiphilic triblock polymer,
where the polymeric block components A and C both include a hydrophilic group and the polymeric block component B includes a hydrophilic group.

Optionally, in the method, the polymeric block components A and C may be both from any one of the following materials: polyethylene glycol, polyvinyl alcohol, polyvinylpyrrolidone, polyether, polyester, polyamide, polypeptide and polysaccharide.

Additionally or alternatively, the polymeric block component B may be from any one of the following materials: polyoxypropylene, polycaprolactone, polylactic acid and poly(lactic-co-glycolic acid).

Optionally, in the method, the polymeric block component A or C may be from a charged hydrophilic polymer.

Optionally, in the method, the ABA-type amphiphilic triblock polymer may be selected from one or more of the following materials: poloxamer and polyethylene glycol-polycaprolactone-polyethylene glycol.

Additionally or alternatively, the ABC-type amphiphilic triblock polymer may be selected from one or more of the following materials: polyethylene glycol-polycaprolactone-glucan and polyethylene glycol-polycaprolactone-polyvinylpyrrolidone.

Optionally, in the method, the stabilizer may be poloxamer. Additionally or alternatively, the contrast agent may be iopamidol. Additionally or alternatively, the drug may include paclitaxel, sirolimus or a derivative thereof

Optionally, in the method, poloxamer and iopamidol may be present at a weight ratio of 1:0.1 to 1:10.

Optionally, in the method, poloxamer and iopamidol may be present at a weight ratio of 1:0.5 to 1:5.

Optionally, in the method, the drug may include a crystalline drug and/or an amorphous drug.

Optionally, in the method, the crystalline drug and the amorphous drug may be present at a weight ratio of 100:0 to 1:99.

Optionally, in the method, the crystalline drug and the amorphous drug may be present at a weight ratio of 70:30 to 100:0.

Optionally, in the method, obtaining the raw material of the drug coating may include the steps of:
obtaining a first solution by dissolving the stabilizer in a first solvent;
obtaining a second solution by dissolving the drug in a second solvent;
obtaining a nano-drug particle suspension by mixing the first solution with the second solution; and
obtaining the raw material of the drug coating by mixing the nano-drug particle suspension with the contrast agent,
wherein the first solvent is water and the second solvent is an organic solvent.

Optionally, in the method, obtaining the raw material of the drug coating may include the steps of:
obtaining a first solution by dissolving the stabilizer in a first solvent;
obtaining a second solution by dissolving the drug in a second solvent; and
obtaining a nano-drug particle suspension as the raw material of the drug coating by mixing the first solution with the second solution,
wherein the first solvent is water and the second solvent is an organic solvent.

Compared with the prior art, the drug coating of the present invention can form a nano-drug particle suspension in a water-soluble environment, thus releasing nano-drug particles. This allows high drug loading and desirable delivery of the drug. In particular, an amphiphilic triblock polymer with hydrophilic segments at both terminals is included in the drug coating as a stabilizer, which enables drug particles in the drug coating to be rapidly restored to the original nano size upon the drug coating coming into contact with water (including blood), almost without any particle size increase. This not only avoids the risk of embolism caused by granules of piled up drug particles, but also enables higher device safety, increased drug uptake and improved therapeutic effects.

The drug coating of the present invention can further include a hydrophilic spacer including a contrast agent and/or a lyoprotectant. Both the contrast agent and the lyoprotectant exhibit desirable hydrophilic properties and can well separate and disperse nano-drug particles in the drug coating and thereby provide hydrophilic spacing between them. This reduces piling up of the nano-drug particles and ultimately facilitates their rapid re-dispersion in an aqueous environment where the drug coating is soluble. As a result, the drug particles can be restored to the original nano size as soon as the drug coating comes into contact with water, almost without any increase in particle size. This additionally reduces the risk of embolism caused by granules of piled up drug particles and enables even higher device safety, even increased drug uptake and even improved therapeutic effects.

In addition to the above drug coating, the drug-loaded medical device or drug balloon of the present invention is further provided with a porous film (or porous film layer) over its surface. This porous film can significantly reduce drug loss during delivery of the medical device, thus allowing a much lower initial drug dose of the drug coating (i.e., the amount of the drug contained in the raw material of the drug coating). This can reduce toxic and side effects of the drug, avoid the development of hemangioma from multiple overlapping proliferations at the lesion, and further increase device safety.

### BRIEF DESCRIPTION OF THE DRAWINGS

FIGS. 1a to 1c are flowcharts of process for preparing a drug coating according to preferred embodiments of the present invention.

FIGS. 2a and 2b are electron microscope images of porous films on drug balloons according to embodiments of the present invention.

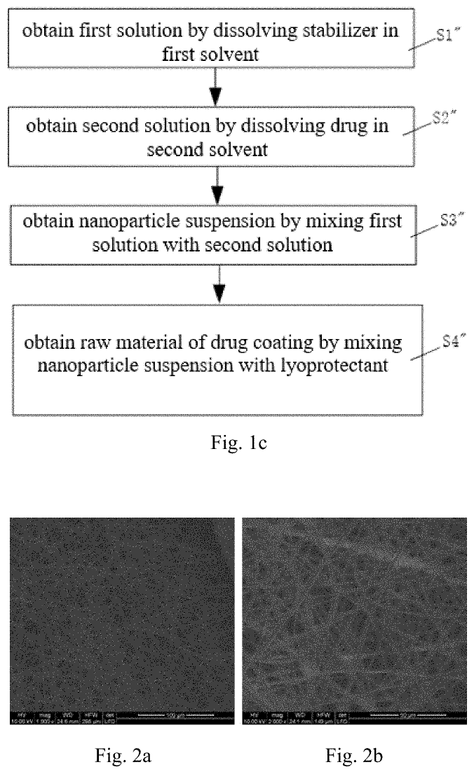

### DETAILED DESCRIPTION

Embodiments of the present invention will be described below by way of particular examples. Based on the disclosure and teachings provided herein, a person of ordinary skill in the art will readily realize other advantages and benefits provided by the present invention. The present invention may also be otherwise embodied or applied through different embodiments, and various modifications or changes may be made to the details disclosed herein from different points of view or for different applications, without departing from the spirit of the present invention. It should be noted that the accompanying drawings are provided herein merely to schematically illustrate the basic concept of the present invention. Accordingly, they only show components relating to the present invention but not necessarily depict all the components as well as their real shapes and dimensions in practical implementations. In practice, the configurations, counts and relative scales of the components may vary arbitrarily and their arrangements may be more complicated.

In the following, each of the embodiments is described as having one or more technical features. However, this does not mean that the present invention must be practiced necessarily with all such technical features, or separately with some or all the technical features in any of the embodiments. In other words, as long as the present invention can be put into practice, a person skilled in the art may choose some or all of the technical features in any of the embodiments or combine some or all of the technical features in different embodiments based on the teachings herein and depending on relevant design specifications or the requirements of practical applications. In this way, the present invention can be carried out more flexibly.

Objectives, features and advantages of the present invention will become more apparent upon reading the following more detailed description of the present invention, which is set forth by way of particular embodiments with reference to the accompanying drawings. Note that the figures are provided in a very simplified form not necessarily drawn to exact scale and for the only purpose of facilitating easy and clear description of the embodiments. As used herein, the singular forms “a”, “an” and “the” include plural referents, unless the context clearly dictates otherwise. As used herein, the phrase “a plurality of” means at least two, unless the context clearly dictates otherwise. As used herein, the term “or” is employed in the sense including “and/or” unless the context clearly dictates otherwise. Additionally, it is to be noted that reference numerals and/or characters may be repeatedly used throughout the embodiments disclosed hereafter. Such repeated use is intended for simplicity and clarity and does not imply any relationship between the discussed embodiments and/or configurations. It is to be also noted that when a component is described herein as being “connected” to another component, it may be connected to the other component either directly or via one or more intervening elements.

As described in the Background section, although nano-drug particles are considered to be ideal for drug coating of drug balloons, nano-drug particles reported so far can rarely be restored to their original nano-sized forms when applied to balloons, aggregate into agglomerates which may pile up into large granules that may fall off and tend to cause embolism, and suffer from low device safety, considerable dug loss during delivery and difficulties in ensuring drug loading.

In order to overcome these problems associated with the conventional nano-drug coatings, the present invention proposes a method of preparing a drug coating. In addition to the ability to prepare a nano-drug coating, this method utilizes an amphiphilic triblock polymer with hydrophilic segments at both terminals in the drug coating as a stabilizer, which allows nano-drug particles to be rapidly restored to their original nano size almost without any particle size increase as soon as the drug coating comes into contact with water. This not only reduces the risk of piling up of drug particles into granules that may cause embolism, but also increases the device's safety. Additionally, an increased amount of drug uptake can be achieved, leading to better therapeutic effects.

Specifically, a drug coating proposed in the present invention includes a stabilizer and a drug. The stabilizer includes an amphiphilic triblock polymer with hydrophilic segments at both terminals. The drug coating can, in a water-soluble environment, form a nanoparticle suspension. It is to be understood that, when exposed to water (including blood), the drug coating can be rapidly dissolved to form a nano-drug particle suspension in which the drug is dispersed in the form of nanoparticles that can be more easily taken up by tissue.

The method includes: obtaining a raw material of the drug coating; and then forming the drug coating by coating the raw material on a surface of a medical device. Here, it would be appreciated that suitable approaches for accomplishing the coating may include, but are not limited to, spraying. The coating may also be accomplished by dipping or otherwise. The raw material of the drug coating includes the stabilizer and the drug, which can form a nano-drug particle suspension in an aqueous environment where the raw material can be dissolved.

The inventors have found that the amphiphilic triblock polymer with hydrophilic segments at both terminals can form a dense hydrophilic layer on the surface of nano-drug particles. Compared with regular amphiphilic diblock polymers, the hydrophilic polymeric segments at both terminals of the amphiphilic triblock polymer present stronger interaction and steric effects, which enable the nanoparticles to have thicker hydrophilic shells. As a result, piling up of the nano-drug particles in the drug coating is reduced, facilitating the restoration of the drug coating to its original nano form and satisfactorily overcoming the problem that conventional nano-drugs can be rarely restored to their nano forms when coated on balloon surfaces. The nano-drug particles are avoided from falling off while being piled up, reducing the risk of embolism and enhancing the device's safety.

Preferably, the drug coating further includes a hydrophilic spacer, which includes a contrast agent and/or a lyoprotectant. The inventors have found that combining the amphiphilic triblock polymer with hydrophilic segments at both terminals with the hydrophilic spacer can impart to the nano-drug coating excellent ability to restore its nano form, thus better overcoming the problem that conventional nano-drug particles can be rarely restored to their nano forms when coating on device surfaces. The nano-drug particles are avoided from falling off while being piled up, effectively reducing the risk of embolism caused by granules that have fallen off and desirably guaranteeing the device's safety.

The contrast agent is implemented primarily as an organic iodine contrast agent, which does not have any toxic or side effect. Moreover, during the preparation of the drug coating, the contrast agent disperses the nano-drug particles and provides hydrophilic spacing between them. Further, the organic iodine contrast agent is a non-ionic contrast agent such as one or more of iohexol, iopamidol, iopromide, ioversol, iodixanol and iotrolan, with iopamidol being more preferred.

In a preferred embodiment of the present invention, the amphiphilic triblock polymer with hydrophilic segments at both terminals is chosen as poloxamer, and the contrast agent as iopamidol. The combined used of poloxamer and iopamidol enables the nano-drug coating to well restore its nano form. Specifically, poloxamer can form a dense hydrophilic layer on the surface of the nano-drug particles in the drug coating, which has stronger steric effects and enables the nano-drug particles to have thicker hydrophilic shells. As a result, piling up of the nano-drug particles is reduced. Meanwhile, iopamidol can separate and disperse the nano-drug particles and thereby provide hydrophilic spacing between them. This additionally prevents aggregation of the nano-drug particles and makes the resulting drug coating loose and porous. In this case, water can penetrate into the drug coating faster through capillary action, and iopamidol can be dissolved into the water as soon as it comes into contact therewith because of its high water solubility, resulting in rapid re-dispersion of the nano-drug particles. Thus, the nano-drug coating on the surface of the drug-loaded medical device (e.g., a drug balloon) can restore its original nano size almost without any particle size increase within only 10 to 40 seconds after its exposure to water. This not only reduces the risk of embolism caused by granules, but also increases the device's safety. Additionally, an increased amount of drug uptake can be achieved, leading to better therapeutic effects.

Additionally, the contrast agent with hydrophilic spacing can be replaced with a lyoprotectant or a mixture of the two. In other words, it is possible either to use the contrast agent or a lyoprotectant alone or to use them in combination. A lyoprotectant, also known as a freeze-drying excipient, is a substance added to a sample undergoing lyophilization in order to enhance the sample's stability during the lyophilization process through providing hydrophilic spacing and maintaining the sample's skeleton. In the present invention, a lyoprotectant is added during the preparation of the drug coating to stabilize the resulting drug coating stable and provide hydrophilic spacing to reduce piling up of the nano-drug particles. The lyoprotectant may be selected from one or more of a saccharide, a polyhydroxy compound, an amino acid, a polymer, an inorganic salt, or the like.

When implemented as a saccharide, the lyoprotectant may be selected from one or more of sucrose, trehalose, mannitol, lactose, glucose and maltose. When implemented as a polyhydroxy compound, the lyoprotectant may be selected from one or more of glycerol, sorbitol, inositol and thiol. When implemented as an amino acid, the lyoprotectant may be selected from one or more of proline, tryptophan, sodium glutamate, alanine, glycine, lysine hydrochloride, sarcosine, L-tyrosine, phenylalanine and arginine. When implemented as a polymer, the lyoprotectant may be selected from one or more of polyvinylpyrrolidone (PVP), gelatin, polyethyleneimine, glucan (or dextran), polyethylene glycol, Tween 80 and bovine serum albumin When implemented as an inorganic salt, the lyoprotectant may be selected from one or more of phosphate, acetate and citrate.

In the present invention, the amphiphilic triblock polymer with hydrophilic segments at both terminals may be an ABA-type amphiphilic triblock polymer, or an ABC-type amphiphilic triblock polymer, or a combination of the two. Preferably, it is poloxamer, which is an ABA-type amphiphilic triblock polymer. Here, the polymeric block components A and C both include hydrophilic groups, and the polymeric block component B includes a hydrophilic group. The hydrophilic group functions to enable the amphiphilic triblock polymer with hydrophilic segments at both terminals to absorb on the surface of the nanoparticles to stabilize the drug (i.e., serving as a stabilizer).

In addition, the polymeric block component A may be from any of the following materials: polyethylene glycol, polyvinyl alcohol, polyvinylpyrrolidone, polyether, polyester, polyamide, polypeptide and polysaccharide. The polymeric block component C may be from any of the following materials: polyethylene glycol, polyvinyl alcohol, polyvinylpyrrolidone, polyether, polyester, polyamide, polypeptide and polysaccharide.

Further, the polymeric block component B may be from any of the following materials: polyoxypropylene, polycaprolactone (PCL), polylactic acid (PLA) and poly(lactic-co-glycolic acid) (PLGA).

Further, the polymeric block component A or C may be from a charged hydrophilic polymer capable of introducing charge repulsion, which can further reduce piling up of the drug nanoparticles, thus additionally enhancing their dispersion, suppressing their aggregation and improving their restoration to the nano form. Examples of the charged hydrophilic polymer may include, but are not limited to, poloxamer, which not only provides strong steric effects, but is also negatively charged (−20 mV) to enable desirable dispersion of anti-proliferative drug nanoparticles. It is to be understood that more surface charge of the nanoparticles can better facilitate restoration of the drug nanoparticles, making them more easily restorable from the device surface to monodispersed nanoparticles.

Further, a molecular weight ratio of the polymeric block components A, B and C in the ABC-type amphiphilic triblock polymer (calculated according to the molecular formulas of the polymeric block components A, B and C) may be (0.5-3):1:(0.5-3), e.g., (0.5-2.5):1:(0.5-2.5), (1-2.5):1:(1-2.5) or (0.5-2):1:(0.5-2). Preferably, the molecular weight ratio is (1-2):1:(1-2). Additionally, a molecular weight ratio of the polymeric block components A and B in the ABA-type amphiphilic triblock polymer may be (1.0-6):1, e.g., (1.0-5):1, (1.0-4):1 or (2.0-5):1. Preferably, the molecular weight ratio is (2-4):1.

Further, the ABA-type amphiphilic triblock polymer with hydrophilic segments at both terminals may be selected from one or more of the following materials: poloxamer and polyethylene glycol-polycaprolactone-polyethylene glycol (PEG-PCL-PEG). Additionally, the ABC-type amphiphilic triblock polymer may be selected from one or more of the following materials: polyethylene glycol-polycaprolactone-glucan and polyethylene glycol-polycaprolactone-polyvinylpyrrolidone.

It is to be understood that, in the present invention, apart from the amphiphilic triblock polymer with hydrophilic segments at both terminals that serves as a stabilizer, another stabilizer (e.g., an amphiphilic diblock polymer) may be also added to the drug coating. During the obtainment of the raw material of the drug coating, the amphiphilic triblock polymer with hydrophilic segments at both terminals serving as a stabilizer should be included at an amount that is sufficient to ensure that the nano-drug can restore its nano size. In a preferred embodiment, the drug coating includes only one stabilizer consisting of the amphiphilic triblock polymer with hydrophilic segments at both terminals.

Further, the drug is implemented primarily as an anti-proliferative drug for the treatment of various cardiovascular diseases. The anti-proliferative drug is preferred to include paclitaxel, sirolimus or derivatives of paclitaxel and sirolimus (here, the term “derivatives of paclitaxel and sirolimus” refer to derivatives of paclitaxel and derivatives of sirolimus). More preferably, the anti-proliferative drug includes paclitaxel. One of the reasons for this is that paclitaxel is more hydrophilic than sirolimus and can be more easily adhere to the wall of a blood vessel. Moreover, it can be taken up and maintain an effective therapeutic concentration for an extended period of time. In contrast, sirolimus will be lost rapidly after it is released during expansion and can rarely effectively inhibit the proliferation of vascular smooth muscle cells. Another reason is that it has been found in late follow-up that paclitaxel-coated balloons exert positive remodeling effects on blood vessels, but those coated with sirolimus do not. Therefore, using paclitaxel as an anti-proliferative drug is not beneficial in a late stage.

In a preferred embodiment of the present invention, the drug coating includes paclitaxel, poloxamer, iopamidol and the lyoprotectant. Preferably, poloxamer and iopamidol (or another organic iodine contrast agent) are present in the drug coating at a weight ratio ranging from 1:0.1 to 1:10 (e.g., 1:0.2 to 1:9, 1:0.3 to 1:8, 1:0.4 to 1:7, 1:0.5 to 1:6), with 1:0.5 to 1:5 being more preferred.

Further, the drug in the drug coating may include a crystalline drug, a non-crystalline (or amorphous) drug, or a combination of the two. Additionally, the crystalline drug and the amorphous drug may be present at a weight ratio of 100:0-1:99, e.g., 50:50-100:0, 80:20-100:0, 90:10-100:0, 70:30-100:0 or 60:40-100:0, with 70:30-100:0 being more preferred. Here, the drug is preferred to be crystalline, because the crystalline form enables better retention of the drug and longer maintenance an effectively drug concentration in tissue. Methods available for the preparation of the crystalline nano-drug are majorly nano-precipitation, ultrasonic preparation and high-pressure homogenization. All of them are conventional techniques and, therefore, need not be described in further detail herein.

The present invention also provides a drug-loaded medical device with a drug coating on its surface. The drug-loaded medical device can be used either in vivo or in vitro. Moreover, it is suitable for both short-term use and long-term permanent implantation. Possible implementations of the drug-loaded medical device of the present invention include, but are not limited to, stents and balloons. In some embodiments, the drug-loaded medical device is a drug balloon.

In addition, in order to avoid loss during delivery, the drug-coated surface is preferably covered with a porous film (or porous film layer). The porous film may be prepared by an electrospinning technique. Such an electrospun film (i.e., a film prepared by electrospinning) will not bring damage to the drug coating and can be easily made at various thicknesses and with various pore sizes. The thickness of the film will not lead to an increase in the device's size and can thus facilitate its delivery. In particular, considering that if the nano-drug coating is dissolved in blood during delivery, it will easily transform back to nanoparticles and washed away by blood, covering the drug coating with the electrospun film can significantly reduce loss of the nano-drug coating during delivery and ensure sufficient drug loading. Moreover, since the electrospun film is a porous structure, it can ensure that the nano-drug particles can flow out after expansion of the device through micropores in the film In addition, as the nano-drug coating is not in direct contact with the wall of a blood vessel, friction between them is avoid, additionally reducing loss during delivery. It is to be understood that most loss of a drug balloon occurs during its delivery. The present invention can greatly reduce loss during delivery and thereby achieve the same tissue drug concentration and therapeutic effect at a lower dose of the drug with less toxic and side effects. As a result, hemangioma and other complications that may develop from multiple overlapping proliferations at the lesion can be avoided, resulting in increased safety. The electrospinning may be solution electrospinning or melt electrospinning The thickness of the porous film should not be too large or too small. An excessive thickness will lead to an increase in the device's size, which is unfavorable to delivery. On the other hand, the film would be ineffective in blocking loss of the drug if its thickness is too small. For these reasons, the thickness of the porous film is preferred to lie in the range of 1 μm to 100 μm. Further, the porous film may have a pore size between 1 μm and 50 μm. The porous film may cover the drug coating. Alternatively and reversely, the drug coating may cover the porous film

Further, the porous film is preferred to include a first layer and a second layer. The first layer is disposed external to the drug coating, and the second layer is disposed external to the first layer. More preferably, the first layer is made of a material selected from one or more of polyurethane, high internal phase emulsion foam, nylon and silk fibroin, and/or the second layer is made of a material selected from one or more of PTFE or a hydrophilic polymer. This can reduce friction between the porous film and the wall of a blood vessel, suppressing resistance during delivery. In order to reduce loss during delivery, a porous matrix layer of polytetrafluoroethylene (PTFE) and/or a hydrophilic polymer may be formed using electrospinning over a surface of the porous film.

The present invention is not limited to any particular size of the nanoparticles, and the size of them may be the same as that of conventional nano-drug particles, such as 1-1000 nm, preferably 3-300 nm, more preferably 50-250 nm. The nano-drug particles are not limited to having any particular shape, and for example, they may assume the shape of spheres, bars, worms or discs, with spheres being more preferred. Drug loading of the nano-drug particles may be 1-99%, preferably 50-80%.

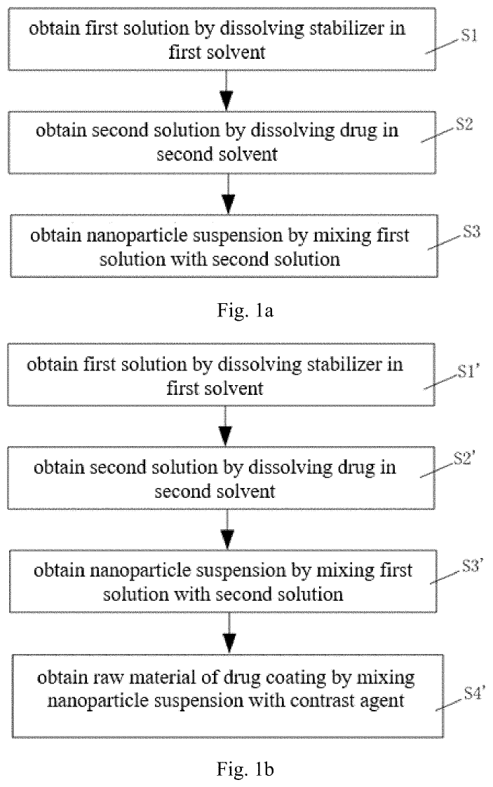

As noted above, in embodiments of the present invention, the raw material of the drug coating may include a stabilizer and a drug. As shown in FIG. 1a, in this case, a process for preparing the raw material of the drug coating may include the steps of:

(S1) dissolving the stabilizer in a first solvent to obtain a first solution;

(S2) dissolving the drug in a second solvent to obtain a second solution; and

(S3) mixing the first solution with the second solution to obtain a nanoparticle suspension as the raw material of the drug coating.

The first solvent may be pure water, ethanol, ethyl acetate, chloroform or the like, without limiting the present invention in any way. Any solvent in which the stabilizer can be dissolved is possible. In some embodiments, the first solvent is an aqueous solvent. Examples of the second solvent may include, but are not limited to, acetone and other organic solvents such as ethanol, methanol and dimethyl sulfoxide. The second solvent should be chosen as being miscible with the first solvent. In some embodiments, the second solvent is preferred to be an organic oil-phase solvent. Steps S1 and S2 may be conducted either simultaneously or successively.

In another embodiment, the raw material of the drug coating may include a stabilizer, a drug and a contrast agent. As shown in FIG. 1b, in this case, a process for preparing the raw material of the drug coating may include the steps of:

(S1′) dissolving the stabilizer in a first solvent to obtain a first solution;

(S2′) dissolving the drug in a second solvent to obtain a second solution;

(S3′) mixing the first solution with the second solution to obtain a nanoparticle suspension; and

(S4′) mixing the nanoparticle suspension with the contrast agent to obtain the raw material of the drug coating.

Steps S1′ and S2′ may be conducted either simultaneously or successively. Moreover, without limitation, the preparation of the nanoparticle suspension may include dialysis in a dialysis bag.

In yet another embodiment, the raw material of the drug coating may include a stabilizer, a drug and a lyoprotectant. As shown in FIG. 1c, in this case, a process for preparing the raw material of the drug coating may include the steps of:

(S1″) dissolving the stabilizer in a first solvent to obtain a first solution;

(S2″) dissolving the drug in a second solvent to obtain a second solution;

(S3″) mixing the first solution with the second solution to obtain a nanoparticle suspension; and

(S4″) mixing the nanoparticle suspension with the lyoprotectant to obtain the raw material of the drug coating.

Likewise, Steps S1″ and S2″ may be conducted either simultaneously or successively.

The preparation of the drug coating and drug-loaded medical device of the present invention will be explained in greater detail with reference to Embodiments 1 to 11 below. Although the following description is set forth in the context of the drug-loaded medical device being implemented as a drug balloon as an example, this should not be construed as limiting the present invention in any sense.

Embodiment 1

In this embodiment, a drug coating was prepared using nano-precipitation, as detailed below.

First of all, poloxamer 188 (as a stabilizer) was fully dissolved in pure water (first solvent; the pure water is as defined in the pharmacopoeia) at 25° C. to produce an aqueous poloxamer solution (first solution) with a concentration of 0.15% (weight ratio w/w). Paclitaxel (anti-proliferative drug) was dissolved in acetone (second solvent) to produce a solution of paclitaxel in acetone (second solution), in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer.

The mixed solution of paclitaxel and poloxamer was then placed in a dialysis bag and dialyzed therein for 12 h in water which was exchanged every 2 h. As a result, a nanoparticle suspension was obtained. Here, the dialysis was carried out to further remove the organic solvent so that the resulting nanoparticle suspension did not contain acetone. It is to be understood that the nanoparticle suspension was a mixture of fine, solid nano-drug particles and the liquid in which they were suspended. The nanoparticle suspension was then concentrated for subsequent use. A Malvern ZS90 instrument was used to measure the size and surface charge of nano-drug particles in the nanoparticle suspension (at a temperature of 2° C. and a scattering angle of 90°, with water being used as a dispersion medium). A measurement was conducted on a high-performance liquid chromatography (HPLC) system (mobile phase: methanol/acetonitrile/water=23:36:41; column temperature: 30° C.; detection wavelength: 227 nm; injection volume: 10 μL), and drug loading was then calculated therefrom.

Next, the nanoparticle suspension was mixed with iopamidol (hydrophilic spacer) at a weight ratio of 1:1 (w/w, by the weight of paclitaxel; i.e., at a 1:1 weight ratio of paclitaxel to iopamidol), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide. Amorphous nano-drug particles were prepared through the above process steps.

Embodiment 2

This embodiment differs from Embodiment 1 in preparing crystalline nano-drug particles through the following process steps.

At first, poloxamer 188 was fully dissolved in pure water at 3° C. to produce an aqueous poloxamer solution with a concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution, with a temperature of the solution being maintained as not exceeding 4° C. using a using an ice-water bath. Stirring was continued for 5 min at speed of 500 rpm to cause evaporation of acetone. As a result, a mixed solution of paclitaxel and poloxamer was obtained.

Subsequently, the resulting mixed solution of paclitaxel and poloxamer was transferred into an ultrasonic cell disintegrator, where it was subjected to ultrasonic waves for 20 min. The ultrasonic waves were delivered at 400 W in 5-s cycles separated by 3-s intervals, with the solution being kept in an ice-water bath and thereby maintained at a temperature not exceeding 3° C. As a result of the ultrasonic pulverization process, a nanoparticle suspension with crystalline nano-drug particles was obtained. After that, the nanoparticle suspension was placed in a dialysis bag and dialyzed therein for 12 h in water which was exchanged every 2 h. The dialyzed nanoparticle suspension was concentrated for subsequent use. A Malvern ZS90 instrument was used to measure the size and surface charge of the nano-drug particles in the nanoparticle suspension. A measurement was conducted on an HPLC system, and drug loading was then calculated therefrom.

Next, the nanoparticle suspension was mixed with iopamidol at a weight ratio of 1:1 (w/w, by the weight of paclitaxel; i.e., at a 1:1 weight ratio of paclitaxel to iopamidol), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μum and an average pore size of 20 μm was formed over the drug coating by electrospinning, followed by sterilization with ethylene oxide.

Embodiment 3

Differing from Embodiment 1, trehalose was used as a hydrophilic spacer in the drug coating prepared in this embodiment.

Specifically, poloxamer 188 was first fully dissolved in pure water at 25 ° C. to produce an aqueous poloxamer solution with a poloxamer 188 concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer.

The mixed solution of paclitaxel and poloxamer was then placed in a dialysis bag and dialyzed therein for 12 h in water which was exchanged every 2 h. As a result, a nanoparticle suspension was obtained. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading was then calculated therefrom.

Next, the nanoparticle suspension was mixed with trehalose at a weight ratio of 1:1 (w/w, by the weight of paclitaxel; i.e., at a 1:1 weight ratio of paclitaxel to trehalose), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide.

Embodiment 4

Differing from Embodiment 1, mannitol was used as a hydrophilic spacer in the drug coating prepared in this embodiment.

Specifically, poloxamer 188 was first fully dissolved in pure water at 25 ° C. to produce an aqueous poloxamer solution with a poloxamer 188 concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer.

The mixed solution of paclitaxel and poloxamer was then placed in a dialysis bag and dialyzed therein for 12 h in water which was exchanged every 2 h. As a result, a nanoparticle suspension was obtained. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading was then calculated therefrom.

Next, the nanoparticle suspension was mixed with mannitol at a weight ratio of 1:1 (w/w, by the weight of paclitaxel; i.e., at a 1:1 weight ratio of paclitaxel to mannitol), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide.

Embodiment 5

Differing from Embodiment 1, sodium glutamate was used as a hydrophilic spacer in the drug coating prepared in this embodiment.

Specifically, poloxamer 188 was first fully dissolved in pure water at 25 ° C. to produce an aqueous poloxamer solution with a poloxamer 188 concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer.

The mixed solution of paclitaxel and poloxamer was then placed in a dialysis bag and dialyzed therein for 12 h in water which was exchanged every 2 h. As a result, a nanoparticle suspension was obtained. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading

Next, the nanoparticle suspension was mixed with sodium glutamate at a weight ratio of 1:1 (w/w, by the weight of paclitaxel; i.e., at a 1:1 weight ratio of paclitaxel to sodium glutamate), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide.

Embodiment 6

Differing from Embodiment 1, glucan was used as a hydrophilic spacer in the drug coating prepared in this embodiment.

Specifically, poloxamer 188 was first fully dissolved in pure water at 25° C. to produce an aqueous poloxamer solution with a poloxamer 188 concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer.

The mixed solution of paclitaxel and poloxamer was then placed in a dialysis bag and dialyzed therein for 12 h in water which was exchanged every 2 h. As a result, a nanoparticle suspension was obtained. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading

Next, the nanoparticle suspension was mixed with glucan at a weight ratio of 1:1 (w/w, by the weight of paclitaxel; i.e., at a 1:1 weight ratio of paclitaxel to glucan), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide.

Embodiment 7

Differing from Embodiment 1, a citrate was used as a hydrophilic spacer in the drug coating prepared in this embodiment.

Specifically, poloxamer 188 was first fully dissolved in pure water at 25 ° C. to produce an aqueous poloxamer solution with a poloxamer 188 concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer.

The mixed solution of paclitaxel and poloxamer was then placed in a dialysis bag and dialyzed therein for 12 h in water which was exchanged every 2 h. As a result, a nanoparticle suspension was obtained. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading was then calculated therefrom.

Next, the nanoparticle suspension was mixed with a citrate at a weight ratio of 1:1 (w/w, by the weight of paclitaxel; i.e., at a 1:1 weight ratio of paclitaxel to the citrate), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide.

Embodiment 8

Differing from Embodiments 1 and 2, the drug coating prepared in this embodiment did not contain iopamidol.

Specifically, poloxamer 188 was first fully dissolved in pure water at 25 ° C. to produce an aqueous poloxamer solution with a poloxamer 188 concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer

The mixed solution of paclitaxel and poloxamer was then placed in a dialysis bag and dialyzed therein for 12 h in water which was exchanged every 2 h. As a result, a nanoparticle suspension was obtained. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading was then calculated therefrom.

An ultrasonic spraying device was then used to spray the nanoparticle suspension onto a balloon surface until drug loading on the balloon surface reached 1.5 μg/mm². Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide.

Embodiment 9

Differing from Embodiment 1, an amphiphilic diblock polymer rather than an amphiphilic triblock polymer with hydrophilic segments at both terminals was used as a stabilizer in the drug coating prepared in this embodiment. Moreover, the vitamin amphiphilic diblock polymer, E polyethylene glycol succinate, was chosen as the stabilizer, and iopamidol as a contrast agent, in this embodiment.

First of all, vitamin E polyethylene glycol succinate (TPGS) was fully dissolved in pure water at 25° C. to produce an aqueous TPGS solution with a concentration of 0.15% (w/w). Paclitaxel was dissolved in acetone to produce a solution of paclitaxel in acetone, in which paclitaxel was present at a concentration of 40 mg/mL.

The solution of paclitaxel in acetone was then added to the aqueous TPGS solution. In this process, the solution of paclitaxel in acetone might be added under stirring at a volume ratio of 1:10 (v/v) of the solution of paclitaxel in acetone to the aqueous TPGS solution. Stirring was continued for evaporation of acetone, resulting in a mixed solution of paclitaxel and TPGS.

The mixed solution of paclitaxel and TPGS was then placed in a dialysis bag and dialyzed therein for 12 h in water which was exchanged every 2 h. As a result, a nanoparticle suspension was obtained. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading was then calculated therefrom.

Next, the nanoparticle suspension was mixed with iopamidol at a weight ratio of 1:1 (w/w, by the weight of paclitaxel; i.e., at a 1:1 weight ratio of paclitaxel to iopamidol), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Additionally, an elastic, porous polyurethane film with a thickness of 20 μm and an average pore size of 20 μm was formed over the drug coating by electrospinning, followed by sterilization with ethylene oxide.

Embodiment 10

Differing from Embodiment 1, no porous film was formed on the drug coating prepared in this embodiment through the process steps described below.

First of all, poloxamer 188 was fully dissolved in pure water at 25 ° C. to produce an aqueous poloxamer solution with a concentration of 0.15% (weight ratio w/w). Paclitaxel was dissolved in acetone to produce a solution of paclitaxel in acetone, in which paclitaxel was present at a concentration of 40 mg/mL.

The solution of paclitaxel in acetone was then added to the aqueous poloxamer solution. In this process, the solution of paclitaxel in acetone might be added under stirring at a volume ratio of 1:10 (v/v) of the solution of paclitaxel in acetone to the aqueous poloxamer solution. Stirring was continued for evaporation of acetone, resulting in a mixed solution of paclitaxel and poloxamer.

The mixed solution of paclitaxel and poloxamer was then placed in a dialysis bag and dialyzed therein for 12 h in water which was exchanged every 2 h. As a result, a nanoparticle suspension was obtained as a mixture of fine, solid particles and the liquid in which they were suspended. The resulting nanoparticle suspension was then concentrated for subsequent use. A Malvern ZS90 instrument was used to measure the size and surface charge of nano-drug particles in the nanoparticle suspension. A measurement was conducted on an HPLC system, and drug loading was then calculated therefrom.

Next, the nanoparticle suspension was mixed with iopamidol at a weight ratio of 1:1 (w/w, by the weight of paclitaxel; i.e., at a 1:1 weight ratio of paclitaxel to iopamidol), and the mixture was then subjected to ultrasonic waves to achieve uniform dispersion. An ultrasonic spraying device was then used to spray the nanoparticle suspension onto a balloon surface until drug loading on the balloon surface reached 1.5 μg/mm² Subsequently, it was subjected to natural drying for 24 h and stored for subsequent use. In this way, a drug coating was formed on the balloon surface.

Embodiment 11

In this embodiment, nano-drug particle size and surface charge measurements were conducted on the drug balloons prepared in Embodiments 1 to 9 using a Malvern ZS90 instrument. Moreover, surface drug loading of the drug balloons prepared in Embodiments 1 to 9 was measured using an HPLC system. The results are summarized in Table 1.

TABLE 1
Characterization of Nano-drug Particles

| | Particle | Polydis- | | Drug Loading |
|---|---|---|---|---|
| | Size of | persity | Surface | on Balloon |
| | Nanoparticles | Index | Charge | Surface |
| | (nm) | (PDI) | (mV) | % (w/w) |
| Embodiment 1 | 233.3 ± 8.6 | 0.175 | −21 | 59% |
| Embodiment 2 | 228.6 ± 4.7 | 0.134 | −26 | 60% |
| Embodiment 3 | 237.8 ± 3.3 | 0.195 | −20 | 62% |
| Embodiment 4 | 234.2 ± 4.6 | 0.210 | −23 | 60% |
| Embodiment 5 | 235.4 ± 6.1 | 0.176 | −20 | 58% |
| Embodiment 6 | 242.9 ± 5.7 | 0.169 | −21 | 59% |
| Embodiment 7 | 230.2 ± 2.6 | 0.188 | −20 | 61% |
| Embodiment 8 | 234.7 ± 12.3 | 0.172 | −19 | 43% |
| Embodiment 9 | 264.2 ± 6.2 | 0.208 | −12 | 41% |

As shown in Table 1, all the nano-drug particles prepared in Embodiments 1 to 9 have a particle size of less than 300 nm, indicating that the drug coating proposed in the present invention is suitable for the transport of nano-drugs. In addition, the nano-drug particles prepared in Embodiments 1 to 8 have surface charge of -19 mV or greater, while those prepared in Embodiment 9 have surface charge of -12 mV. All of them exhibit good stability. Additionally, drug loading of all the nano-drug particles prepared in these embodiments is 40% (w/w) or higher. Compared with conventional drug coatings, a lower initial drug dose is allowed, resulting in less toxic and side effects.

Further, tests were performed to evaluate restoration of the drug balloons prepared in Embodiments 1 to 9 to their nano forms. Specifically, the drug balloons prepared in Embodiments 1 to 9 were inflated and submerged in pure water at 37 ° C. for 60 seconds, and a Malvern ZS90 instrument was then used to measure the sizes of drug particles in the water. The results are summarized in Table 2.

TABLE 2
Restoration of Drug Coatings to Nano Forms

| | Initial Nano-drug Particles | | Restored Nano-drug Particles | |
|---|---|---|---|---|
| | Particle Size | Polydispersity | Particle Size | Polydispersity |
| | (nm) | Index (PDI) | (nm) | Index (PDI) |
| Embodiment 1 | 233.3 ± 8.6 | 0.175 | 257.8 ± 2.6 | 0.188 |
| Embodiment 2 | 228.6 ± 4.7 | 0.134 | 250.1 ± 7.6 | 0.201 |
| Embodiment 3 | 237.8 ± 3.3 | 0.195 | 260.2 ± 7.2 | 0.223 |
| Embodiment 4 | 234.2 ± 4.6 | 0.210 | 266.7 ± 8.3 | 0.202 |
| Embodiment 5 | 235.4 ± 6.1 | 0.176 | 270.1 ± 6.1 | 0.215 |
| Embodiment 6 | 242.9 ± 5.7 | 0.169 | 275.3 ± 10.9 | 0.243 |
| Embodiment 7 | 230.2 ± 2.6 | 0.188 | 252.8 ± 7.5 | 0.232 |
| Embodiment 8 | 234.7 ± 12.3 | 0.172 | 265.1 ± 8.1 | 0.226 |
| Embodiment 9 | 264.2 ± 6.2 | 0.208 | Particles larger | — |
| | | | than 1 micron, | |
| | | | which are visible | |
| | | | by naked eye | |

As shown in Table 2, both the drug coatings prepared in Embodiments 1 to 2 can be rapidly restored to particle sizes similar to those of the initial nano-drug particles, with particle size increases of only 20 nm to 30 nm, demonstrating that the combined use of poloxamer and iopamidol enables excellent restoration of the drug coating to their original nano forms, as indicated by the very small polydispersity indices (PDI). The drug coatings prepared in Embodiments 3 to 7 in which lyoprotectants are used as hydrophilic spacers can also be rapidly restored to particle sizes similar to those of their initial nano-drug particles, with particle size increases of only 20 nm to 30 nm, indicating that these drug coatings containing lyoprotectants as hydrophilic spacers also have excellent ability to restore their nano forms. Additionally, although without hydrophilic spacing provided by iopamidol, the drug coating of Embodiment 8 can also desirably restore its nano form. In contrast, due to lacking steric effects provided by an amphiphilic triblock polymer with hydrophilic segments at both terminals, the drug coating prepared on the drug balloon in Embodiment 9 using an amphiphilic diblock polymer failed to restore its initial nano form and was observed with peeling off in the form of lumps of particles, which tend to cause embolism. It is to be understood that nano-drug particles with a smaller PDI can be dispersed more uniformly and provide better drug transport.

Further, tests were also carried out to evaluate loss during delivery of the drug balloons prepared in Embodiments 1 to 10. Specifically, each of the drug balloons prepared in Embodiments 1 to 10 was inserted into an in vitro vascular model without being inflated, with a time taken to reach a target being controlled to 60 s. After the balloon was withdrawn, an HPLC system was used to measure an amount of the drug remaining on the surface of the drug balloon, and a drug loss rate during delivery was derived therefrom. The results are summarized in Table 3.

TABLE 3
Drug Loss Rates during Delivery of Drug balloons

| | Drug Loss Rate during Delivery |
|---|---|
| Embodiment 1 | 3.5% |
| Embodiment 2 | 4% |
| Embodiment 3 | 3.1% |
| Embodiment 4 | 2.8% |
| Embodiment 5 | 3% |
| Embodiment 6 | 3.5% |
| Embodiment 7 | 3.6% |
| Embodiment 8 | 2.1% |
| Embodiment 9 | 1.6% |
| Embodiment 10 | 37% |

Further, tests were performed on the drug balloons prepared in Embodiments 1 to 10 to evaluate uptake in tissue. Isolated porcine arterial segments were kept constant at 37° C., and sterilized bare balloons were placed therein to dilate the vessel segments at 6 atm for 1 min. After depressurization, the bare balloons were withdrawn. The drug balloons prepared in the Embodiments were placed in the dilated vessel segments to again dilate them at 6 atm for 1 min. After depressurization, the drug balloons were withdrawn Immediately after that, they were washed 3 times with 1 mL each time of a phosphate-buffered saline (PBS) solution. Tissue drug concentrations were measured using a gas chromatography-mass spectrometry (GC-MS) instrument, and amounts of the drug remaining on the surface of the drug balloons were measured using an HPLC instrument. The results are summarized in Table 4.

TABLE 4
Immediate Drug Concentrations in Tissue
Resulting from Drug balloons

| | Immediate Drug Concentration | | Drug Remaining on |
|---|---|---|---|
| | in Tissue (ng/mg) | | Balloon Surface (%) |
| Embodiment 1 | 549.2 ± 117 | ng/mg | 4% |
| Embodiment 2 | 477.1 ± 65 | ng/mg | 3% |
| Embodiment 3 | 522.3 ± 112 | ng/mg | 3% |
| Embodiment 4 | 468.3 ± 89 | ng/mg | 4.6% |
| Embodiment 5 | 553.9 ± 135 | ng/mg | 4% |
| Embodiment 6 | 492.4 ± 80.3 | ng/mg | 2.5% |
| Embodiment 7 | 506.5 ± 56 | ng/mg | 3% |
| Embodiment 8 | 207.1 ± 12 | ng/mg | 8% |
| Embodiment 9 | 75.4 ± 49 | ng/mg | 28% |
| Embodiment 10 | 85.3 ± 43 | ng/mg | 0.4% |

As can be seen from Tables 3 and 4, due to the absence of a porous film, the balloon prepared in Embodiment 10 was observed with high loss of the drug during delivery (37%). In contrast, the values of this parameter for those prepared in Embodiments 1 to 9 were extremely low (1-4%). Therefore, the presence of a porous film can effectively reduce drug loss during delivery, resulting in good performance during use. As can be seen from the immediate drug concentrations in tissue, due to the excellent ability to restore the original nano forms and low loss during delivery, the balloons of Embodiment 1 to 7 created very high tissue concentrations, with small amounts of the drug remaining on the balloon surface, suggesting good drug transport performance. Although lacking hydrophilic spacing provided by iopamidol, Embodiment 8 also showed good ability to restore the nano form and low loss during delivery. Moreover, the drug concentration in tissue was also high, and there was a small amount of the drug remaining on the balloon surface. However, due to failure to be restored to the initial nano-drug particles, drug particles in Embodiment 9 aggregated into big lumps, which were less favorable to uptake by tissue and led to a relatively low immediate drug concentration in tissue. Due to absence of a porous film and hence significant loss during delivery, Embodiment 10 showed a low drug concentration in tissue.

Therefore, it has been experimentally proven that the nano-drug coatings prepared in accordance with the present invention, which each contain an amphiphilic triblock polymer with hydrophilic segments at both terminals as a stabilizer, can be rapidly restored to their original nano sizes upon coming into contact with water, almost without any increase in particle size. This not only avoids the risk of embolism caused by granules, but also enables higher device safety, increased drug uptake and improved therapeutic effects. In particular, when added with a hydrophilic spacer, the nano-drug coatings can be even better restored to their nano forms. In particular, the drug coatings are each covered with a porous film, which can greatly reduce drug loss during delivery of the medical device and result in a higher immediate drug concentration in tissue and hence improved drug transport.

The description presented above is merely that of a few preferred embodiments of the present invention and is not intended to limit the scope thereof in any sense. Any and all changes and modifications made by those of ordinary skill in the art based on the above teachings fall within the scope of the invention.

1. A drug-loaded medical device, wherein a surface of the drug-loaded medical device has a drug coating, the drug coating comprising a stabilizer and a drug, the stabilizer comprising an amphiphilic triblock polymer with hydrophilic segments at both terminals, the drug coating forming a nano-drug particle suspension in a water-soluble environment.

2. The drug-loaded medical device according to claim 1, wherein the drug coating further comprises a hydrophilic spacer, the hydrophilic spacer comprising a contrast agent and/or a lyoprotectant.

3. The drug-loaded medical device according to claim 2, wherein the contrast agent is selected from one or more of iohexol, iopamidol, iopromide, ioversol, iodixanol and iotrolan, and
the lyoprotectant is selected from one or more of a saccharide, a polyhydroxy compound, an amino acid, a polymer and an inorganic salt.

4. The drug-loaded medical device according to claim 3, wherein the saccharide is selected from one or more of sucrose, trehalose, mannitol, lactose, glucose and maltose,
the polyhydroxy compound is selected from one or more of glycerol, sorbitol, inositol and thiol,
the amino acid is selected from one or more of proline, tryptophan, sodium glutamate, alanine, glycine, lysine hydrochloride, sarcosine, L-tyrosine, phenylalanine and arginine,
the polymer is selected from one or more of polyvinylpyrrolidone, gelatin, polyethyleneimine, glucan, polyethylene glycol, Tween 80 and bovine serum albumin, and
the inorganic salt is selected from one or more of a phosphate, an acetate and a citrate.

5. The drug-loaded medical device according to claim 1, wherein the amphiphilic triblock polymer with hydrophilic segments at both terminals is an ABA-type amphiphilic triblock polymer and/or an ABC-type amphiphilic triblock polymer,
where the polymeric block components A and C both comprise a hydrophilic group and the polymeric block component B comprises a hydrophilic group.

6. The drug-loaded medical device according to claim 5, wherein the polymeric block components A and C are both from any one of the following materials: polyethylene glycol, polyvinyl alcohol, polyvinylpyrrolidone, polyether, polyester, polyamide, polypeptide and polysaccharide, and/or
wherein the polymeric block component B is from any one of the following materials: polyoxypropylene, polycaprolactone, polylactic acid and poly(lactic-co-glycolic acid); and
wherein the ABA-type amphiphilic triblock polymer is selected from one or more of the following materials: poloxamer and polyethylene glycol-polycaprolactone-polyethylene glycol, and/or
wherein the ABC-type amphiphilic triblock polymer is selected from one or more of the following materials: polyethylene glycol-polycaprolactone-glucan and polyethylene glycol-polycaprolactone-polyvinylpyrrolidone.

7. (canceled)

8. The drug-loaded medical device according to claim 1, further comprising a porous film layer covering the drug coating.

9. A drug balloon, comprising a balloon body and, provided on a surface of the balloon body, a drug coating and a porous film layer, the drug coating comprising a stabilizer and a drug, the stabilizer comprising an amphiphilic triblock polymer with hydrophilic segments at both terminals, the drug coating forming a nano-drug particle suspension in a water-soluble environment.

10. The drug balloon according to claim 9, wherein the drug coating further comprises a hydrophilic spacer, the hydrophilic spacer comprising a contrast agent and/or a lyoprotectant.

11. The drug balloon according to claim 10, wherein the stabilizer is poloxamer, and/or the contrast agent is iopamidol, and/or the drug comprises paclitaxel, sirolimus or a derivative thereof paclitaxel and sirolimus, and/or wherein the lyoprotectant comprises one or more of a saccharide, a polyhydroxy compound, an amino acid, a polymer and an inorganic salt.

12. The drug balloon according to claim 11, wherein the saccharide is selected from one or more of sucrose, trehalose, mannitol, lactose, glucose and maltose, the polyhydroxy compound is selected from one or more of glycerol, sorbitol, inositol and thiol,
the amino acid is selected from one or more of proline, tryptophan, sodium glutamate, alanine, glycine, lysine hydrochloride, sarcosine, L-tyrosine, phenylalanine and arginine,
the polymer is selected from one or more of polyvinylpyrrolidone, gelatin, polyethyleneimine, glucan, polyethylene glycol, Tween 80 and bovine serum albumin, and
the inorganic salt is selected from one or more of a phosphate, an acetate and a citrate.

13. A method of preparing a drug-loaded medical device, comprising:
obtaining a raw material of a drug coating, the raw material of the drug coating comprising a stabilizer and a drug, the stabilizer and the drug forming a nano-drug particle suspension in a water-soluble environment;
preparing the drug-loaded medical device by forming the drug coating on a surface of a medical device using the raw material of the drug coating; and
providing a porous film layer on a surface of the drug coating.

14. A method of preparing a drug coating, comprising:
obtaining a raw material of the drug coating, the raw material of the drug coating comprising a stabilizer and a drug, the stabilizer and the drug forming a nano-drug particle suspension in a water-soluble environment; and
forming the drug coating on a surface of a medical device using the raw material of the drug coating,
wherein the stabilizer comprises an amphiphilic triblock polymer with hydrophilic segments at both terminals.

15. The method according to claim 14, wherein the raw material of the drug coating further comprises a hydrophilic spacer, the hydrophilic spacer comprising a contrast agent and/or a lyoprotectant.

16. The method according to claim 15, wherein the contrast agent is selected from one or more of iohexol, iopamidol, iopromide, ioversol, iodixanol and iotrolan, and
the lyoprotectant is selected from one or more of a saccharide, a polyhydroxy compound, an amino acid, a polymer and an inorganic salt.

17. The method according to claim 16, wherein the saccharide is selected from one or more of sucrose, trehalose, mannitol, lactose, glucose and maltose,
the polyhydroxy compound is selected from one or more of glycerol, sorbitol, inositol and thiol,
the amino acid is selected from one or more of proline, tryptophan, sodium glutamate, alanine, glycine, lysine hydrochloride, sarcosine, L-tyrosine, phenylalanine and arginine,
the polymer is selected from one or more of polyvinylpyrrolidone, gelatin, polyethyleneimine, glucan, polyethylene glycol, Tween 80 and bovine serum albumin, and
the inorganic salt is selected from one or more of a phosphate, an acetate and a citrate.

18. The method according to claim 14, wherein the amphiphilic triblock polymer with hydrophilic segments at both terminals is an ABA-type amphiphilic triblock polymer and/or an ABC-type amphiphilic triblock polymer,
where the polymeric block components A and C both comprise a hydrophilic group and the polymeric block component B comprises a hydrophilic group.

19. The method according to claim 18, wherein the polymeric block components A and C are both from any one of the following materials: polyethylene glycol, polyvinyl alcohol, polyvinylpyrrolidone, polyether, polyester, polyamide, polypeptide and polysaccharide, and/or
wherein the polymeric block component B is from any one of the following materials: polyoxypropylene, polycaprolactone, polylactic acid and poly(lactic-co-glycolic acid); or
wherein the polymeric block component A or C is from a charged hydrophilic polymer.

20. (canceled)

21. The method according to claim 18, wherein the ABA-type amphiphilic triblock polymer is selected from one or more of the following materials: poloxamer and polyethylene glycol-polycaprolactone-polyethylene glycol, and/or
wherein the ABC-type amphiphilic triblock polymer is selected from one or more of the following materials: polyethylene glycol-polycaprolactone-glucan and polyethylene glycol-polycaprolactone-polyvinylpyrrolidone.

22. The method according to claim 14, wherein the drug comprises a crystalline drug and/or an amorphous drug.
